(19) United States
(12) Patent Application Publication
(10) Pub. No.: US 2016/0008274 A1
(43) Pub. Date: Jan. 14, 2016

(54) TREATMENT OF NAIL DISORDERS

(71) Applicant: Francisco TAUSK, Pittsford, NY (US)

(72) Inventor: Francisco TAUSK, Pittsford, NY (US)

(73) Assignee: University of Rochester, Rochester, NY (US)

(21) Appl. No.: 14/326,186

(22) Filed: Jul. 8, 2014

(51) Int. Cl. A61K 9/00; A61K 31/496; A61K 31/517; A61K 31/4196; A61K 31/427; A61K 31/56; A61K 38/08; A61K 31/192; A61K 38/13; A61K 31/436; A61K 31/519; A61K 31/4174; A61K 31/5375

(52) U.S. Cl. CPC A61K 9/0014; A61K 31/4174; A61K 31/496; A61K 31/517; A61K 31/4196; A61K 31/427; A61K 31/5375; A61K 38/08; A61K 31/192; A61K 38/13; A61K 31/436; A61K 31/519; A61K 31/56

(57) ABSTRACT

A nail disorder can be treated using a device, such as a pad or covering, that delivers a drug for treating the disorder. The pad can be secured to skin over at least a portion of the nail matrix and extend over less than one-third of the nail plate. When applied, the drug carried by the pad can penetrate into the skin until reaching the nail matrix, where the nail plate is formed. The drug can treat nail disorders such as onychomycosis or psoriasis.

### BACKGROUND

1. Field of the Inventions

The present disclosures relate to the treatment of nail disorders, such as onychomycosis and psoriasis. More specifically, the present disclosures relate to treatment devices and methods for treating nail disorders.

2. Description of the Related Art

Infectious nail disorders can be caused by a number of agents, including molds, fungi, and yeast organisms.

Onychomycosis has long been recognized as a difficult nail disorder to treat. The lengthy period over which the nail takes to grow, the hardness of the nail plate, the location of the infectious process between the nail bed and plate are major factors for difficulty in treatment. Current treatments for these conditions are primarily oral anti-infective agents that are lipophilic and have poor nail penetration if applied topically. Examples of these agents include terbinafine, itraconazole, and fluconazole.

### SUMMARY

In accordance with some embodiments disclosed herein is the realization that systemic treatments have numerous associated drawbacks. For example, these treatments can last from about three to six months because the nails grow very slowly. Further, these treatments require the incorporation of drugs to the nail matrix in order to be incorporated into the growing nail plate. These treatments are also associated with significant potential systemic drug interactions and side effects, such as systemic toxicities, requiring periodic laboratory blood tests. In order to avoid systemic toxicities, it is important to develop methods to treat these local disorders using local anti-infective agents. Such treatments can provide significant improvements to current therapies, including less toxicity and potentially greater efficacy.

However, the use of topical drugs to directly treat the nail has also been ineffective. For example, topical drugs like ciclopirox (an antifungal drug) have only been marginally successful because of its poor penetration across the layers of the nail. Indeed, the nail is a highly keratinized membrane, which is a formidable barrier to diffusion. Traditionally, penetration of the nail using oral antifungal drugs in topical formulations has not worked well.

A variety of methods are being developed in order to increase nail penetration, such as a lacquer that is currently available in the market, or other interventions currently being developed, such as the use of iontophoresis, other electrical currents, and penetration enhancers. However, an aspect of at least some of the embodiments disclosed herein is the realization that the downside of all of these treatments is that they target the nail plate, and not specifically the nail matrix, therefore they do not incorporate antifungal drug to the area where the nail is being formed.

Therefore, according to some embodiments disclosed herein, a treatment device or medication delivery system can be used to treat nail disorders at the location where the nail is formed. For example, in some embodiments, a topical treatment can be provided by which medication is incorporated to the nail matrix thereby allowing such medication to be incorporated to the nail plate and protecting the nail plate from infection.

In accordance with some embodiments, various devices and methods for treating nail disorders are provided herein. For example, a method of treating a digit having a nail disorder, wherein the digit comprising a nail plate and a nail matrix, can comprise: providing a device, such as a pad or covering, having a drug for treating the disorder; and securing the pad to skin over at least a portion of the nail matrix and to extend over less the entirety of the nail plate.

In some embodiments, the method can comprise: providing a pad having a drug for treating the disorder, the pad comprising an arcuate edge; and applying the pad to a digit, the digit comprising a nail plate, a nail cuticle, and a nail matrix, the pad being applied proximal to the nail plate such that (i) an edge of the pad extends along an arcuate edge of the nail cuticle and (ii) the pad overlies the nail matrix. In some embodiments, edge of the pad can be arcuate and extend along the cuticle.

In some implementations of the method, when the digit further comprises a nail cuticle, the pad can be positioned entirely proximal of the nail cuticle. For example, the pad can comprise an arcuate edge, and pad can be positioned such that the arcuate edge is positioned adjacent to the nail cuticle. Further, the digit can further comprise a distal interphalangeal joint, and the nail matrix can be located generally proximal of the nail cuticle and distal of the distal interphalangeal joint. The pad can be positioned entirely distal of the interphalangeal joint.

The pad can comprise a drug-containing portion, and securing the pad can comprise securing the pad with the drug-containing portion being disposed proximal to a nail cuticle. The drug-containing portion comprise an anti-inflammatory drug, an antifungal drug, or other active agents that can treat a nail disorder. In some embodiments, the pad can further comprise a drug-free periphery portion, and the pad can be secured with the periphery portion being disposed proximal to a nail cuticle.

In accordance with some embodiments, pad can be secured to the skin to overlie less than one-half, one-third, one-fourth, one-fifth, or less of the nail plate. For example, the pad can cover less than a 3 mm length, to millimeter length, or a 1 mm length of the nail plate. Further, when a lunula of the digit is visible beneath the nail plate, the pad can be secured to the digit such that the pad overlies at least a portion, such as a majority or the entirety, of the lunula.

The methods and devices disclosed herein can be used to treat various nail disorders, such as bacterial infections, fungal infections, and other disorders, such as onychomycosis, pseudomonas, nail psoriasis, paronychia, or other fungal or yeast infections or other ailments disclosed herein or known in the art that may affect the nails. Notably, between 5% and 12% of the general population of the United States suffers from onychomycosis, which is the most common nail disorder. Further, approximately 3% of the population suffers from psoriasis, and frequently, these patients have disease in their nails.

The device can comprise anything that can be applied, such as a pad, sponge, bandage, dressing, patch, coating, or other covering. In some embodiments, the device 100 can comprise micro needles. Further, the device can comprise a liquid or gel applied to skin adjacent or proximal to the nail cuticle using a brush, dipping technique, or other coating method. Further, in some embodiments, the device can comprise a material backing and a drug reservoir.

In some embodiments, the device can comprise first and second components that each having a drug portion disposed thereon. The drug portion of the first component can have one or more different properties than the drug portion of the second component. The first component can be placed, for example, over the nail plate, and the second component can be placed over the skin of the finger, such as over the nail matrix. The drug portion of the first component can be configured to treat nail plate differently than the drug portion of the second component.

For example, the drug portion of the first component can comprise a different dosage, formulation, strength, placement, or drug than the drug portion of the second component.

Additionally, in some embodiments, the second component can extend along an edge of the first component. For example, the first component can be generally rectangular, square, or trapezoidal, and the second component can extend along a single side thereof and be positionable over the nail matrix while the first portion is positioned on top of the nail plate.

Further, the second component can extend about the first component along opposing lateral edge thereof and along an edge extending between the opposing lateral edges. For example, the first component can be generally rectangular, square, or trapezoidal, and the second component can extend along three sides thereof and be positionable over the nail matrix and lateral sides of the nail (on top of the lateral nail folds), while the first portion is positioned on top of the nail plate.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 is a side cross-sectional view of a finger and a dispensing device, according to some embodiments.

FIGS. 2A-2E are schematic views of treatment devices, according to some embodiments.

FIGS. 3A-3D are top views of treatment devices positioned on a finger, according to some embodiments.

FIGS. 4A-4B are top views of treatment devices positioned on a finger, according to some embodiments.

### DETAILED DESCRIPTION

While the present description sets forth specific details of various embodiments, it will be appreciated that the description is illustrative only and should not be construed in any way as limiting. Additionally, it is contemplated that although particular embodiments of the present inventions may be disclosed or shown in the context of treating fungal infections in nails, such embodiments can be used in treating bacterial infections and disorders, such as or onychomycosis, pseudomonas, nail psoriasis, paronychia, or other fungal or yeast infections or other ailments disclosed herein or known in the art that may affect the nails. Furthermore, various applications of such embodiments and modifications thereto, which may occur to those who are skilled in the art, are also encompassed by the general concepts described herein.

In accordance with some embodiments, a device for treating nail disorders can be provided that advantageously targets a body system or area responsible for the growth of the nail, similar to the principle of treatment for systemic administration of drugs, but without the negative consequences and side effects associated with systemic administration. For example, a device for treating nail disorders can be attached to the area of skin overlying the nail matrix to deliver a drug or medication that will treat the nail disorder. The device can thereby apply a drug, such as an antifungal or antibacterial drug, to the nail matrix, the area where the nail is being formed and thus incorporate the drug into the nail plate as it grows.

In some embodiments, the treatment device can be attached or adhered directly to the skin adjacent to the nail. The treatment device can be positioned directly over the nail matrix. Further, in some embodiments, the nail plate can be fully exposed. Such embodiments may be more aesthetically pleasing during treatment and avoid conditions that could lead to the growth of bacterial or yeast infections. However, the nail plate can also be partially or fully covered. For example, in some embodiments, the treatment device can cover less than one-half, one-third, one-fourth, one-fifth, or less of the nail plate.

The device can comprise anything that can be applied, such as a pad, sponge, bandage, dressing, patch, coating, or other covering. In some embodiments, the device can comprise micro needles. Further, the device can comprise a liquid or gel applied to skin adjacent or proximal to the nail cuticle using a brush, dipping technique, or other coating method. Further, in some embodiments, the device can comprise a material backing and a drug reservoir.

In accordance with some embodiments, methods of treatment are also provided in which a patient or professional applies a treatment device, such as a pad, containing the drug, to a location directly superficial to a nail matrix. The device can be worn by the patient for a period of time sufficient to allow the drug to penetrate into the nail matrix or sufficient to allow positive results to be seen in the growing nail. For example, depending on the rate of release of the drug, such pads could be replaced daily or weekly.

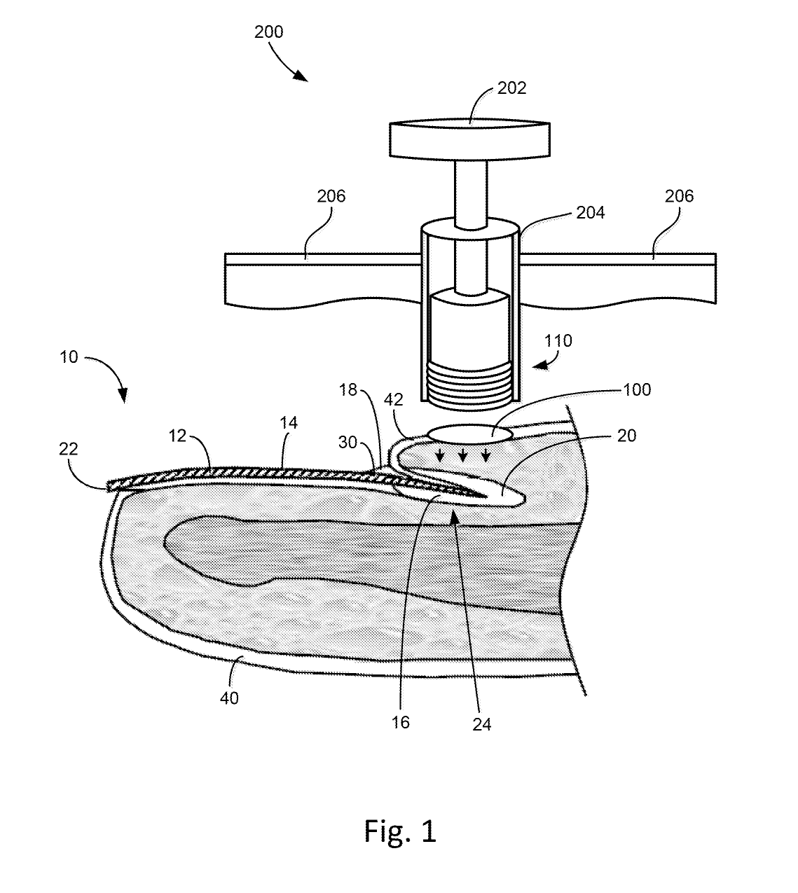

Referring now to the figures, FIG. 1 illustrates a side cross-sectional view of a finger 10 having a nail plate 12. The finger comprises a nail bed 14, in nail matrix 16, a cuticle 18, a nail root 20, and a free margin 22. The nail matrix 16 is a layer of cells at a proximal end 24 of the nail bed 14 (opposite the free margin 22) that rapidly divides and fills with the protein keratin, a structural protein critical to the formation of the nail plate 12. Generally, the nail matrix 16 of a nail plate 12 of a finer can grow at a rate of about 3 mm per month, whereas the nail plate of a toe growing at about one-fourth that rate.

As also illustrated, the finger 10 can also comprise a lunula 30 at the proximal end 24 of the nail plate 12. The lunula 30 can often be seen in top view of the nail plate 12 as a generally crescent-shaped whitish area of the nail matrix 16.

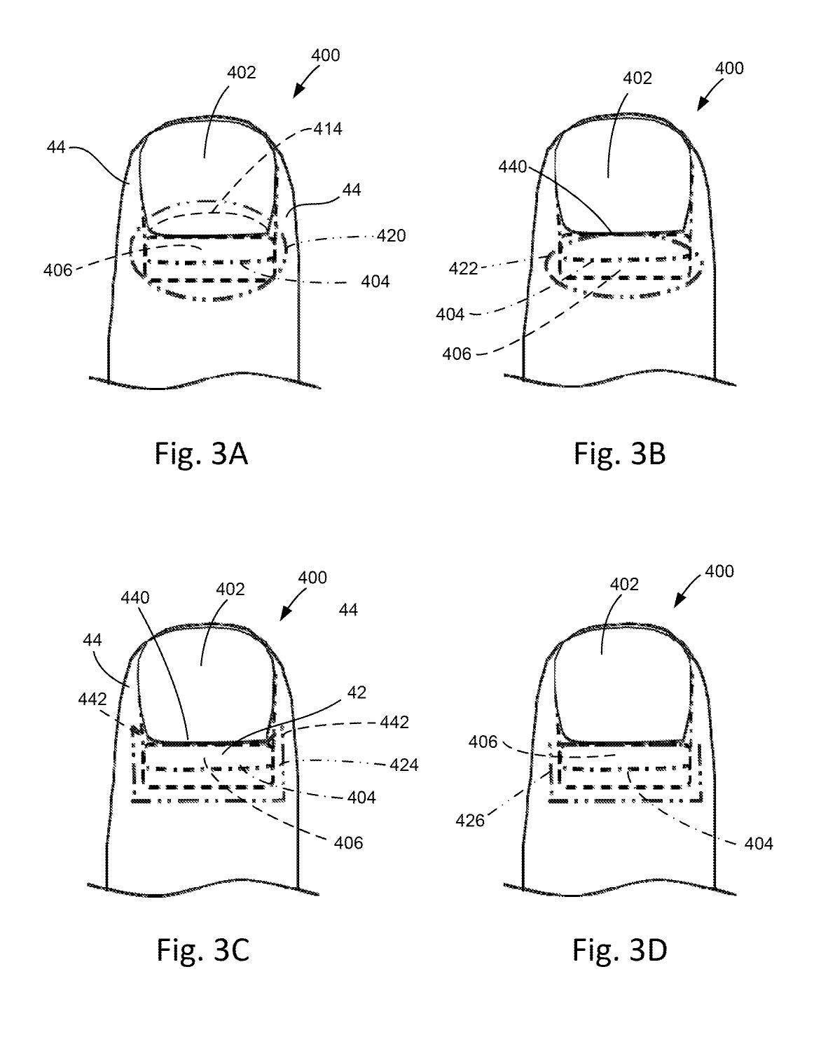

Furthermore, the finger 10 can also comprise skin 40 overlying the tissue of the finger 10. The skin 40 can comprise a proximal nail fold 42 (shown FIG. 1) and lateral nail folds 44 (shown in FIG. 3A). The proximal nail fold 42 comprises the portion of the skin 40 adjacent to the cuticle 18 that overlies the nail matrix 16.

In accordance with some embodiments, a delivery system 200 can be used to apply the device 100 onto the skin overlying the area of the nail matrix 16. For example, FIG. 1 also illustrates the placement of a treatment device 100 onto the proximal nail fold 42, which can be dispensed using a delivery system 200. The delivery mechanism can comprise a plunger component 202, a carrier receptacle 204, and at least one handle component 206. In some embodiments, the plunger component 202 can be spring-loaded in order to facilitate dispensing of each device 100. The delivery system 200 can be configured to carry a plurality of devices 110. For example, the carrier receptacle 204 can enclose or house the plurality of devices 110.

The device 100 can comprise a pad or material configured to deliver a drug that can be absorbed through the skin overlying the area of the nail matrix 16 in order to treat a nail disorder. In some embodiments, the device 100 can comprise an adhesive pad or material that can be impregnated or otherwise carry a drug. Once placed onto the skin overlying the nail matrix 16, the device 100 can remain in place until the drug has been sufficiently absorbed through the skin (shown as the proximal nail fold 42).

For example, the device 100 can comprise a backing, cover, or membrane and an adhesive, such as a pressure sensitive adhesive. The backing of the treatment device can comprise materials such as vinyl, polyethylene, and/or polyester films. The adhesive can comprise a pressure sensitive adhesive that helps in maintaining an intimate contact between transdermal system and the skin surface. The adhesive can allow the treatment device to adhere with application of finger pressure. The adhesive can allow the treatment device to be aggressively and permanently tachy and exert a strong holding force without altering drug release. Additionally, the adhesive can allow the device to be removable from a smooth surface without leaving a residue. The smooth surface can be a mounting surface that can carry the device or to which the device can be coupled until it is removed for application to the patient. The adhesive can comprise suitable biocompatible materials, such as polyacrylates, polyisobutylene and silicon-based adhesives.

The device 100 can be coated, impregnated, or configured to support an amount of a drug. In some embodiments, the device 100 can comprise a drug reservoir or polymer matrix wherein the drug can be carried. Further, in some embodiments, the device 100 can optionally comprise a rate-limiting membrane from which the drug can be diffused to the stratum corneum of the skin, to penetrate the epidermis, and be taken up by the dermis until final delivery or absorption to the nail matrix 16. As the drug is incorporated into the nail matrix 16, the drug will also be incorporated into the nail plate 12 as it grows outwardly from the nail matrix 16.

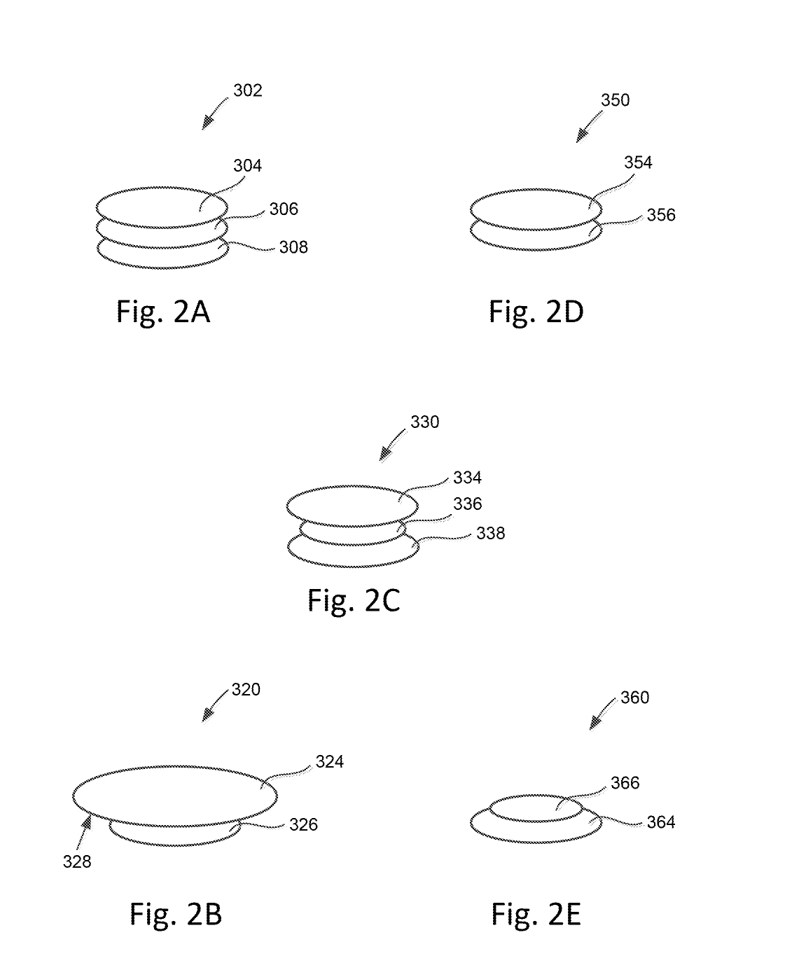

FIGS. 2A-2D illustrate embodiments of the device 100 in which different combinations of backings, drug delivery means, and adhesive means are provided.

For example, FIG. 2A illustrates a treatment device 302 having a nonadhesive backing 304, a drug reservoir or polymer matrix 306, and a porous adhesive 308. The components of the device 302 can be integrated into a single, flat pad that can be adhesively attached to a patient's digit (e.g., a finger or toe).

In some embodiments, the drug reservoir 306 can comprise a matrix or absorbent material that supports, holds, or absorbs an amount of drug for diffusion through the skin toward the nail matrix.

The drug can comprise any of a variety of drugs, including antifungal or anti-inflammatory drugs. Antifungal drugs that can be used with the device include, for example, terbinafine, miconazole, clotrimazole, bifonazole, butoconazole, econazole, fenticonazole, ketoconazole, oxiconazole, sulconazole, albaconazole, fluconazole, itraconazole, ravuconazole, amorolfine, butenafine, naftifine, andilafungin, caspofungin, micafungin, benzoic acid, ciclopirox, tolnaftate, undecylenic acid, crystal violet, or any combination of the aforementioned. Anti-inflammatory drugs can also be used with the device. Such anti-inflammatories can include as corticosteroids, cyclosporine, tacrolimus, pimecrolimus, or methotrexate.

In some embodiments, the drug reservoir 306 can comprise a rate-limiting component for controlling release of the drug from the device. For example, a polymer matrix can be prepared by dispersion of a drug in liquid or solid state into a polymer base. Such polymers can comprise natural or synthetic polymers.

For example, natural polymers can include any of a variety of biocompatible natural polymers, such as cellulose derivatives, zein, gelatin, shellac, waxes, gums, natural rubber and chitosan. Further, synthetic polymers can include any of a variety of biocompatible synthetic polymers, such as polybutadiene, hydrin rubber, polyisobutylene, silicon rubber, nitrile, acrylonitrile, neoprene, butylrubber, polyvinyl alcohol, polyvinylchloride, polyethylene, polypropylene, polyacrylate, polyamide, polyurea, polyvinylpyrrolidone, or polymethylmethacrylate. In some embodiments, porous silicon can be advantageously used due to its properties, which may facilitate retention of a drug and time release of that drug. Furthermore, other polymers can be used, such as cross-linked polyethylene glycol, eudragits, ethyl cellulose, polyvinylpyrrolidone, or hydroxypropylmethylcellulose can be used.

The drug reservoir 306 can be prepared by dissolving a drug and a polymer in a common solvent. The drug should be homogenously dispersed in a hydrophilic or lipophilic polymer. A plasticizer such as dibutylpthalate, triethylcitrate, polyethylene glycol or propylene glycol and permeation enhancer can then be added and mixed into the drug and polymer mixture. The medicated polymer formed thereby can then be mounted onto an occlusive base or substrate, for example, in a compartment or depression of a backing. The backing can comprise a drug impermeable material. Thereafter, an adhesive polymer can then be spread along the circumference of the backing

Additionally, the drug can be mixed with the polymer by homogenously mixing finely ground drug particles with a liquid polymer or a highly viscous base polymer followed by cross linking of polymer chains or homogenously blending drug solids with a rubbery polymer at an elevated temperature. Advantages of matrix pads include absence of dose dumping, direct exposure of polymeric matrix to the skin, and no interference of adhesive.

FIG. 2B illustrates an embodiment in which a treatment device 320 comprises an adhesive backing 324 and a drug reservoir or polymer matrix 326. The adhesive backing 324 can be configured with a larger outer profile or diameter than the drug reservoir 326 such that the drug reservoir 326 can be adhesively coupled to a bottom side or periphery portion 328 of the adhesive backing 324 with a portion of the bottom side 328 being exposed to permit adhesion of the device 320 to a digit of the patient. For example, after the reservoir 326 is coupled to the backing 324, the periphery portion 328 can be coated with an adhesive that permits adhesion of the device 320 to the digit.

In accordance with some embodiments, FIG. 2C illustrates a device 330 comprising a backing 334, a drug reservoir 336, and a rate-limiting membrane 338. The rate-limiting membrane 338 can be nonporous so that the drug is released by diffusing directly through the material, or the material may contain fluid filled micropores in which case the drug may additionally diffuse through the fluid, thus filling the pores. In the case of nonporous membrane 338, the rate of passage of drug molecules depends on the solubility of the drug in the membrane 338 and the thickness of membrane 338. Hence, the choice of material for the membrane 338 can be dependent on the type of drug being used. By varying the composition and thickness of the membrane 338, the dosage rate per unit area of the device can be controlled. Mostly EVA, ethyl cellulose, silicon rubber and polyurethanes are used to prepare the rate-limiting membrane 338. EVA can allow the permeability of the membrane 338 to be altered by adjusting vinyl acetate content of polymer. Polyurethane membranes may be suitable for hydrophobic polar compounds having low permeability through hydrophobic polymers such as silicon rubber or EVA membrane.

The rate-limiting membrane 338 can be prepared by a solvent evaporation method or a compression method. In the solvent evaporation method, a polymer is dissolved in solvent with or without plasticizer. The solution is then poured on a horizontal surface and left for evaporation of solvent in order to obtain a thin film. In the compression method, a polymer can be compressed with the required force at high temperature for a specific period of time.

In some embodiments, a liquid formulation of a drug reservoir can be replaced with a solid polymer matrix (e.g., polyisobutylene), which can be sandwiched between rate-limiting membrane and backing laminate. Further, in some embodiments, the drug reservoir can be formed by suspending a drug solid in an aqueous solution of water miscible drug solubilizer (e.g., polyethylene glycol). The drug suspension can be homogenously dispersed by a high shear mechanical force in lipophilic polymer, forming thousands of unleachable microscopic drug reservoirs (micro reservoirs). The dispersion can be quickly stabilized by immediately cross linking the polymer chains in-situ which produces a medicated polymer layer of a specific area and fixed thickness.

For example, FIG. 2D illustrates another treatment device 350 comprising a nonadhesive backing 354 and an adhesive drug layer 356. The adhesive drug layer can comprise a drug that is incorporated into an adhesive, which can serve as a binder as well as an adhesive mechanism for adhering the device 350 to the digit of the patient.

FIG. 2E illustrates yet another embodiment in which a treatment device 360 comprises an adhesive substrate 364 onto which a drug 366 can be applied. In such an embodiment, the adhesive substrate 364 can comprise a permeable membrane that can be attached to the digit of the patient. Thereafter, the drug 366 can be applied directly to the adhesive substrate 364. The drug 366 can be in a diffuse form that allows the drug 366 to be absorbed into the adhesive substrate 364, such as a liquid, gel, or otherwise.

In accordance with some embodiments, the device can also comprise a permeation enhancer or accelerant. The composition of the skin differs significantly from that of the nails. Penetration through the nail is much more difficult than through the skin, and the vehicles that function as penetration enhancers for nail penetration are often different than those used for skin penetration.

For example, the skin has a high lipid content, whereas the nail is mostly composed of hard keratin with a high content of sulfur, a low content of lipids (about 0.5%), and a low content of water (about 18%). Because of nail's low lipid content, transdermal enhancers, which target disrupting lipid layers, are unsuccessful in penetrating the nail. Therefore, while lipophilic compounds are favorable for skin penetration, hydrophilic materials are favorable for nail penetration. Additionally, physical agents that disrupt the stratum corneum, such as microneedles and hollow needles, would be more favorable for skin penetration rather than nail penetration.

However, some penetration enhancers can work for both skin and nail, such as dimethyl sulfoxide (“DMSO”), urea and salicylic acid. As such, in some embodiments, one or more of DMSO (e.g., between about 10% and about 50%), urea (e.g., between about 10% and about 40%), or salicylic acid (e.g., between about 2% and about 40%) can be used.

Some embodiments can use such permeation enhancers in combination with one or more of the antifungal drug(s) disclosed herein, whether for targeting skin and/or nail penetration.

Examples of penetration enhancers for the skin, which can alter the protein or lipid packaging of stratum corneum, include: solvents, such as methanol, ethanol, dimethyl sulfoxide, propylene glycol, 2-pyrrolidone, isopropyl myristate, laurocapram; surfactants, such as sodium lauryl sulfate, sorbitan monolaurate, pluronic; essential oils, such as cardamom oil, caraway oil, lemon oil, menthol, d-limonene, and linoleic acid, or physical agents, such as pads containing microneedles that penetrate the epidermis or pads containing hollow microneedles that further allow the delivery of the drug directly to the epidermis.

In some embodiments, transdermal penetration enhancers can comprise: glyceryl monoesters; glyceryl monolaurate; terpenes; fatty acids; fatty acid esters; fatty acid alcohols; fatty alcohol derivatives; aliphatic esters; aromatic esters; carboxylic acids; carboxylic esters; alcohols; polyol; triethylcitrate; triethylene glycol; diethylene glycol monoethyl ether; dodecyl-2-N,N-dimthylaminopropionate; diethylphthalate; oleic acid; propylene glycol; lauryl alcohol; ethyl acetate; laurylpyrrolidone; triethanolamine; glycerine; triacetine; diethyleneglycol monoethylether; triacetin; N-methyl-pyrrolidone; lauryl lactate; propylene glycol monolaurate; or diethyleneglycol monoethyl ether.

Further, in some embodiments, transdermal penetration enhancers can comprise polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; tocopheryl polyethylene glycol succinates; sugar esters; sugar ethers; sucroglycerides, and mixtures thereof. In one variation, the hydrophilic surfactant is a tocopheryl polyethylene glycol succinate, e.g., D-alpha-tocopheryl PEG-1000 succinate (vitamin E TPGS).

Suitable nail penetration enhancers are well-known in the art. Because of the significant content of water and protein, most enhancers are directed to softening and hydration, disruption of the disulphide bonds, keratolysis, or cleavage of keratins. Examples of these are: keratolytic agents, 2-mercaptoethanol, sulfites, bisulfites, N-(2-mercaptopropionyl) glycine, N-acetyl-1-cysteine, 2-n-nonyl-1,3-dioxolane, thioglycolic acid, urea-hydrogen peroxide, salicylic acid, urea, papain, keratinase, humectants as sorbitol or glycerol, N-methyl-2-pyrrolidone, polypropylene glycol 400, dimethylsulfoxide, Labrasol, mercaptoethanol, pyrithone.

A keratolytic agent, i.e., a desquamating agent, can help loosen keratin in the nail, thus increasing the nail's permeability and/or aiding in the process of desquamation or removal of the upper layers of the nail. Suitable keratolytic agents include, but are not limited to, urea, ammonium thioglycolate, calcium thioglycolate, potassium thioglycolate, sodium sulfide, benzoylperoxide, salicylic acid, resorcinol, and tretinoin and the like, as well as mixtures thereof. Others can be found in “Remington: The Science and Practice of Pharmacy, Nineteenth Edition, pp. 878-879,” the entirety of which is incorporated herein by reference.

In some embodiments, the keratolytic agent can be present at between about 1% and about 25%, between about 3% to about 18% (w/w), between about 5% and about 20% (w/w), between about 6% and about 30% (w/w), or between about 10 and about 15% (w/w), for example. The keratolytic agent can be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% (w/w).

In some embodiments, the keratolytic agent is present at about 6% (w/w) (e.g., 10% of a 60% aqueous solution of ammonium thioglycolate). Further, in some embodiments, the keratolytic agent can comprise a mixture of urea and ammonium thioglycolate. Urea can be present at about 10% to about 20% (w/w) in the total amount of the mixture of keratolytic agents. That is, even though the amount of keratolytic agent in the composition is even higher (i.e., about 25%, about 30% or about 35% (w/w)), urea can be present from about 10% to about 20% (w/w) of the composition. Alternatively, urea is present at about 15% (w/w) in the mixture of keratolytic agents.

Other physical or penetration enhancers may include devices delivering iontophoresis, electricity, heat, or light. In some embodiments, the permeation enhancer, whether for skin or nail penetration, can be present in an amount of between about 0.1% to about 30% (w/w) of the drug or drug reservoir. The permeation enhancer can be present in an amount of between about 1% and about 15% (w/w), between about 1% and about 10% (w/w), between about 3% and about 15% (w/w), between about 3% and about 10% (w/w), between about 5% and about 15% (w/w) or 5%, between about 10% (w/w). For example, the penetration enhancer can be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% (w/w).

The device can also be configured in a variety of shapes in order to provide sufficient coverage for the nail matrix or other aspects of the nail. For example, referring now to FIGS. 3A-3D, a variety of devices are illustrated that each have a different perimeter profile or shape. Each of FIGS. 3A-3D illustrate a finger 400 having a nail plate 402 that has a proximal edge 404 at which the nail plate 402 is being formed. As discussed above, the nail plate 402 is formed in the nail matrix 406. The various devices shown in FIGS. 3A-3D illustrate shapes and positions of the device relative to the nail plate 402 and the nail matrix 406.

FIGS. 3A-3B illustrate devices 420, 422 that have a generally elliptical perimeter. In FIG. 3A, the device 420 is positioned over the nail matrix 406 and an exposed portion of the nail plate 402. According to some embodiments, the device 420 can be positioned over less than half of the nail plate 402, less than one-third of the nail plate 402, less than one-fourth of the nail plate 402, or less than one-fifth of the nail plate 402.

In some embodiments, the device 420 can be secured to the finger 400 such that the device 420 covers less than a 3 mm longitudinal length of the nail plate 402. Further, the device 420 can cover less than a 2 mm longitudinal length of the nail plate 402. Furthermore, the device 420 can cover less than a 1 mm longitudinal length of the nail plate 402.

As illustrated also in FIG. 3A, the finger 400 can comprise a lunula 414 that is visible underneath the nail plate 402. In some embodiments, the positioning of the device 420 can comprise placing the device 420 such that it overlies at least a portion of the lunula 414. For example, the device 420 can be positioned such that it overlies the majority of or the entirety of the lunula 414.

FIG. 3B illustrates a device 422 positioned over the nail matrix 406, but not overlying the exposed nail plate 402. Accordingly, some embodiments of the device can avoid occlusion or covering of a diseased nail plate, which could otherwise lead to superinfections. An additional advantage can be that such embodiments also avoid the impaired cosmetic appearance of wearing a bandage that covers a large surface of the finger or toe. For example, in some embodiments, as shown in FIG. 3B, the device 422 can be positioned entirely proximal of a nail cuticle 440.

FIG. 3C illustrates a device 424 that has a generally rectangular perimeter with a pair of proximal protrusions 442 that extend in a proximal direction of the device 440. The device 424 is positioned over the nail matrix 406, but does not overlie the exposed nail plate 402. The anterior or proximal edge of the device 424 can comprise one or more straight portions or be configured as an arcuate edge that can complement the acuity of the proximal nail fold 42. For example, the positioning of the device 424 can comprise positioning an arcuate edge adjacent to the nail cuticle 440.

Similarly, FIG. 3D illustrates the device 426 having a substantially rectangular perimeter. The device 426 is positioned over the nail matrix 406, but does not overlie the exposed nail plate 402.

Further, the finger or toe to which the device will be applied can comprise a distal interphalangeal joint, and the nail matrix 406 can be located generally proximal of the nail cuticle 440 and distal of the distal interphalangeal joint. The device can be positioned entirely distal of the interphalangeal joint.

In accordance with some embodiments, the device can be configured to cover multiple aspects of the finger, including the nail, nail matrix, lateral nail folds, and/or cuticle and to comprise components that are separately optimized for treating a given aspect of the finger. In some embodiments, the components can be separately optimized such that the components comprise properties that are different from each other. Such properties can include the amount, strength, or type of drug or treatment material present or supported on the component. Further, the device can be configured to comprise two or more components, according to some embodiments.

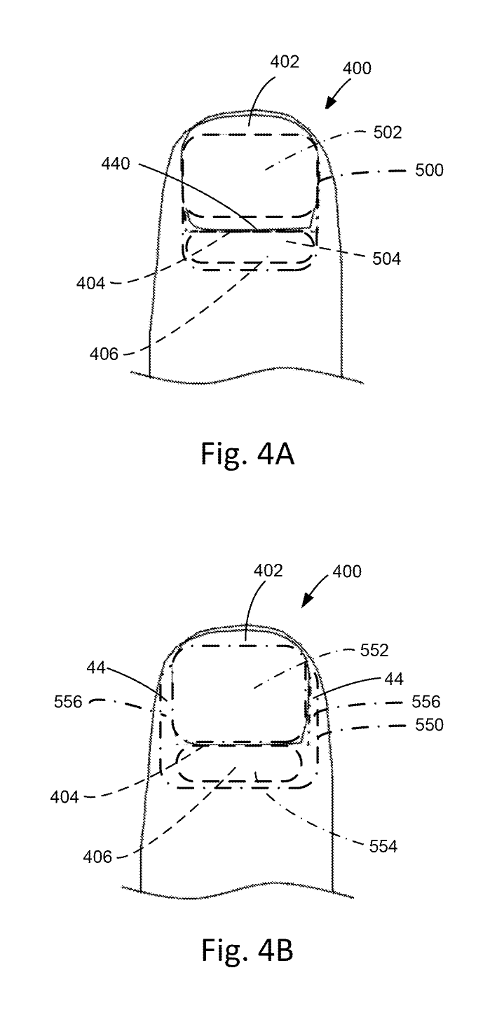

For example, FIGS. 4A-4B illustrate to embodiments of a device that comprises multiple components optimized for treating different aspects of the finger 400. FIG. 4A illustrates a device 500 comprising a first component 502 and a second component 504. The first component 502 can be placed over the nail plate 402, and the second component 504 can be placed over the nail matrix 406.

The first component 502 can comprise a drug or drug reservoir that is optimized to enhance penetration of the drug through the nail plate 402. For example, the type of drug, concentration, or position of the drug as supported by the device 500 can be configured to enhance penetration of the drug through the nail plate 402.

According to some embodiments, the second component 504 can be configured differently from the first component 502 in order to optimize the second component 504 to enhance drug effectiveness and/or cutaneous penetration into and around the nail matrix 406. Thus, the drug concentration, the type of drug, and/or the position of the drug is supported by the device 500 can be configured differently for the second component 504 than for the first component 502.

In FIG. 4B, a device 550 is illustrated that comprises a first component 552 and a second component 554. As in the embodiment shown in FIG. 4A, the first component 552 can be configured to provide an optimize penetration through the nail plate 402. However, in the device 550, the second component 554 can be configured to provide not only enhanced cutaneous penetration into and around the nail matrix 406, but the second component 554 can also comprise lateral portions 556 that extend along the lateral nail folds 44 of the finger 500. The lateral portions 556 can be configured to provide the same or different properties as the portion of the second component 554 that overlies the nail matrix 406.

In some embodiments, the second component 554 can comprise generally uniform characteristics or properties across not only the nail matrix 406, but also across the lateral nail folds 44 (via the lateral portions 556). However, in some embodiments, the second component 554 can comprise varying properties that enable the second component 554 to target the nail matrix 406 differently than the lateral nail folds 44. Further, in some embodiments, the properties of portions of the second component 554 can be generally the same as those of the first component 552 or different from those of the first component 552.

Additionally, some embodiments can comprise a drug that is dissolved in dymethil sulfoxide (DMSO) and applied with a brush to the same area of the skin described above. DMSO is a solvent that will allow virtually unlimited absorption of the drug. The only FDA approved use for DMSO to date is Rimso, which consists of 50% DMSO in water instilled in the bladder for the treatment of interstitial cystitis.

Accordingly, embodiments of the system disclosed herein can allow constant delivery of drugs to the area of the nail matrix, where the nail plate is formed. Such systems avoid the problematic side effects of traditional technologies that treat disorders or fungal diseases of the nails. For example, such embodiments avoid the systemic administration of antifungal drugs with its associated side effects, as well as the topical application of antifungal drugs to the nail plate with its drawback of incomplete penetration of the nail plate and failure to reach the nail matrix, the area where the nail is formed and the same area toward which a fungus or other disorder may spread or become incorporated into the growing nail plate. Such embodiments can advantageously avoid these systemic side effects and ineffective drug delivery, at least in part, by targeting the area where the nail plate is being formed, thus incorporating drugs into the nail itself.

In some embodiments, other additives, such as penetration enhancers, can be incorporated into the system in order to enhance the penetration of the drug through the stratum corneum of the skin overlying the nail matrix.

In addition to the treatment of onychomycosis and other disorders, some embodiments can be used to deliver corticosteroids and treat psoriasis of the nail. Accordingly, embodiments of the systems disclosed herein can be widely used due to the substantial number of patients that suffer from nail disorders, such as fungal infections of the nails or nail psoriasis.

Although embodiments of these inventions have been disclosed in the context of certain examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combinations or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions.

What is claimed is:

1. A method of treating a digit having a nail disorder, the digit comprising a nail plate and a nail matrix, the method comprising:
providing a pad having a drug for treating the disorder; and
securing the pad to skin over at least a portion of the nail matrix and to extend over less than one-third of the nail plate.

2. The method of claim 1, wherein the digit further comprises a nail cuticle, wherein securing the pad comprises positioning the pad entirely proximal of the nail cuticle.

3. The method of claim 2, wherein the pad comprises an arcuate edge, and wherein securing the pad further comprises positioning the arcuate edge adjacent to the nail cuticle.

4. The method of claim 2, wherein the digit further comprises a distal interphalangeal joint, the nail matrix located generally proximal of the nail cuticle and distal of the distal interphalangeal joint, wherein securing the pad comprises positioning the pad entirely distal of the interphalangeal joint.

5. The method of claim 1, wherein the pad comprises a drug-containing portion, wherein securing the pad comprises securing the pad with the drug-containing portion being disposed proximal to a nail cuticle.

6. The method of claim 5, wherein the drug-containing portion comprises an anti-inflammatory drug.

7. The method of claim 5, wherein the drug-containing portion comprises an antifungal drug.

8. The method of claim 5, wherein the pad further comprises a drug-free periphery portion, wherein securing the pad comprises securing the pad with the periphery portion being disposed proximal to a nail cuticle.

9. The method of claim 1, wherein the pad is secured to the skin to overlie less than ¼ of the nail plate.

10. The method of claim 1, wherein the pad covers less than a 3 mm length of the nail plate.

11. The method of claim 10, wherein the pad covers less than a 1 mm length of the nail plate.

12. The method of claim 1, wherein the digit comprises a lunula visible beneath the nail plate, and wherein the securing the pad comprises overlying the lunula with the pad.

13. The method of claim 1, wherein the pad comprises first and second components each having a drug portion disposed thereon, wherein the drug portion of the first component has different properties than the drug portion of the second component, and wherein the method comprises placing the first component over the nail plate and the second component over the skin, the drug portion of the first component being configured to treat nail plate differently than the drug portion of the second component.

14. The method of claim 13, wherein the drug portion of the first component comprises a different dosage than the drug portion of the second component.

15. The method of claim 13, wherein the second component extends about the first component along opposing lateral edge thereof and along an edge extending between the opposing lateral edges.

16. A method for treating a nail disorder, the method comprising:
providing a covering having a drug for treating the disorder, the covering comprising an arcuate edge; and
applying the covering to a digit, the digit comprising a nail plate, a nail cuticle, and a nail matrix, the covering being applied proximal to the nail plate such that (i) an edge of the covering extends along an arcuate edge of the nail cuticle and (ii) the covering overlies the nail matrix.

17. The method of claim 16, wherein the covering comprises a liquid or gel applied to skin adjacent to the nail cuticle.

18. The method of claim 16, wherein the covering comprises a pad secured to the digit adjacent the nail plate without overlying the nail plate.

19. The method of claim 18, wherein the pad comprises the edge, wherein the pad edge is arcuate.

20. The method of claim 18, wherein the pad is secured to the skin to overlie less than one-third of the nail plate.
